(19) United States
(12) Patent Application Publication
(10) Pub. No.: US 2018/0360750 A1
(43) Pub. Date: Dec. 20, 2018

(54) COMPOSITION FOR REDUCING MUSCLE CRAMPS

(71) Applicant: Lillian Johnson, Lehigh Acres, FL (US)

(72) Inventor: Lillian Johnson, Lehigh Acres, FL (US)

(21) Appl. No.: 15/624,768

(22) Filed: Jun. 16, 2017

(51) Int. Cl. A61K 9/00; A61K 33/00; A23L 33/00; A23L 2/39; A23L 2/56; A23L 2/52

(52) U.S. Cl. CPC A61K 9/0095; A61K 33/00; A23L 33/40; A23V 2002/00; A23L 2/56; A23L 2/52; A23L 2/39

(57) ABSTRACT

A composition for reducing muscle cramps includes a composition in a ratio by weight between 70% and 85% parts liquid water, between 3% and 7% parts sodium bicarbonate, and between 12% and 25% flavoring. The composition is drank to prevent muscle cramps.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to muscle cramp relieving drinks and more particularly pertains to a new muscle cramp prevention drink for relieving and reducing muscle cramps brought on by physical activity or a sedentary lifestyle.

### BRIEF SUMMARY OF THE INVENTION

An embodiment of the disclosure meets the needs presented above by generally comprising a composition in a ratio by weight between 70% and 85% parts liquid water, between 3% and 7% parts sodium bicarbonate, and between 12% and 25% flavoring. The composition is drank to relieve muscle cramps.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

Not Applicable

### DETAILED DESCRIPTION OF THE INVENTION

With reference now to the below, a new muscle cramp prevention drink embodying the principles and concepts of an embodiment of the disclosure will be described.

As best stated herein, the composition for reducing muscle cramps generally comprises a drink which may be drank by a person to reduce or prevent muscle cramps particularly brought on by physical activity. The composition consists of, generally in a ratio by weight:

70% to 85% liquid water;

3% to 7% sodium bicarbonate; and

12% to 25% flavoring.

The water may comprise any water though it may be preferred that the water has a pH level of between 6.5 and 8.0. The sodium bicarbonate is in a powder form and is commonly referred to as baking soda and is ubiquitous to grocery stores. The flavoring may be any conventional flavoring used to flavor a liquid for consumption. However, it is preferred that the flavoring comprise a fruit flavor and more particularly a sweetened, powdered flavoring. One example of such a flavoring may include Kool-Aid Cherry Drink Mix, available from Kraft Foods located in Northfield, Ill. and commonly sold in grocery stores. This particular flavoring includes sugar, fructose, citric acid, ascorbic acid, vitamin E acetate, artificial flavor and calcium phosphate.

A more particular example of the composition includes by weight percentage:

78% liquid water;

5% sodium bicarbonate; and

17% parts flavoring.

When mixing the composition, one preferred embodiment includes by mass:

235 grams liquid water;

15 grams sodium bicarbonate; and

50 grams of flavoring.

Finally, another embodiment, utilizing English units, may include:

8 fluid ounces of water;

1 tablespoon of sodium bicarbonate; and

4 tablespoons of powder flavoring.

In use, the three ingredients are mixed together and drank. The composition may be drunk, in particular after physical exertion to alleviate muscle cramping due to lack of fluids and electrolytes. The composition may also be taken in to relieve cramping due to a sedentary lifestyle such as might be experienced by elderly persons living in a nursing home.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

1. (canceled)

2. A composition to be ingested for alleviating muscle cramps, said composition consisting of:
in a ratio by weight:
78% liquid water;
5% sodium bicarbonate; and
17% flavoring.

3. The composition according to claim 2, wherein said flavoring is a fruit flavor.

4. The composition according to claim 2, wherein said flavoring comprises a powder.

5. A composition to be ingested for reducing muscle cramps, said composition consisting of:
in a ratio by weight:
between 70% and 85% liquid water;
between 3% and 7% sodium bicarbonate; and
between 12% and 25% flavoring.

6. The composition according to claim 5, wherein said flavoring is a fruit flavor.

7. The composition according to claim 5, wherein said flavoring comprises a powder.
